(19) United States
(12) Patent Application Publication
(10) Pub. No.: US 2026/0248468 A1
(43) Pub. Date: Aug. 27, 2026

(54) A METHOD FOR ORTHO-POSITRONIUM DETECTION AND IMAGING USING A TIME-OF-FLIGHT POSITRON EMISSION TOMOGRAPH

(71) Applicant: Siemens Medical Solutions USA, Inc., Malvern, PA (US)

(72) Inventor: William Steinberger, Knoxville, TN (US)

(21) Appl. No.: 19/160,271

(22) PCT Filed: Jun. 20, 2023
(86) PCT No.: PCT/US2023/068702

§ 371 Date: Aug. 27, 2025

(51) Int. Cl. A61B 6/03; A61B 6/00

(52) U.S. Cl. CPC A61B 6/037; A61B 6/5217; A61B 6/5282

(57) ABSTRACT

A device for measuring the health of a tissue and a method of use. The device includes a plurality of sensors for detecting an event related to decay of a positron emitted from a pharmaceutical radionuclide in the tissue, and a processor. The processor measures a first count rate indicative of three photon emission related to a first decay mode of the positron, measures a second count rate indicative of two photon emission related to a second decay mode of the positron, applies a scatter correction factor to each of the first count rate and the second count rate to account for scattered and attenuated annihilation photons in the tissue, determines a ratio of the first count rate to the second count rate, determine a decay lifetime for ortho-positronium (o-Ps) based on the ratio, and determines the health of the tissue based on the decay lifetime for o-Ps.

### BACKGROUND OF THE INVENTION

### Field of the Invention

The present invention relates generally to Positron Emission Tomography (PET) and, in particular, to a method for imaging a tissue by measuring a decay lifetime for ortho-positronium generated from a positron emitted from a pharmaceutical radiotracer injected into a patient.

### Description of the Related Art

Positron Emission Tomography can be used to diagnose and characterize cancerous tissue. A radiotracer, such as fluorodeoxyglucose (FDG) that contains the positron-emitting radionuclide 18F, is injected into a patient. FDG will uptake in regions of the body that exhibit a higher metabolic rate, which can be an indication of cancer. Once the radiotracer has been distributed in the body, the emitted positrons can then be imaged to isolate regions of interest (ROI) that have a higher metabolic uptake. The positrons emitted in a given tissue or ROI will either undergo direct annihilation with a free electron or bond with an electron to form positronium (Ps). Ps can be either para-positronium (p-Ps), in which spins of the positron and electron are anti-parallel, or ortho-positronium (o-Ps) in which spins of the positron and electron are parallel.

The lifetime of o-Ps is dependent on the material in which it resides. Current processes for measuring the lifetime of o-Ps requires the emission of a prompt gamma ray to mark a start time. However, the vast majority of radiotracers, such as FDG, do not emit a prompt gamma ray. It is therefore desirable to be able to measure the lifetime of Ps without the need for a prompt gamma ray.

### BRIEF SUMMARY OF THE INVENTION

Disclosed herein is a method of measuring a health of a tissue. The method includes introducing a pharmaceutical radionuclide into the tissue, wherein the pharmaceutical radionuclide emits a positron, measuring a first count rate indicative of three photon emission related to a first decay mode of the positron, measuring a second count rate indicative of two photon emission related to a second decay mode of the positron, applying a scatter correction factor to each of the first count rate and the second count rate to account for scattered and attenuated annihilation photons in the tissue, determining a ratio of the first count rate to the second count rate, determining a decay lifetime for ortho-positronium (o-Ps) based on the ratio, and determining the health of the tissue based on the decay lifetime for o-Ps.

Disclosed herein also is a device for measuring the health of a tissue. The device includes a plurality of sensors for detecting an event related to decay of a positron emitted from a pharmaceutical radionuclide in the tissue, and a processor. The processor is configured to measure a first count rate indicative of three photon emission related to a first decay mode of the positron, measure a second count rate indicative of two photon emission related to a second decay mode of the positron, apply a scatter correction factor to each of the first count rate and the second count rate to account for scattered and attenuated annihilation photons in the tissue, determine a ratio of the first count rate to the second count rate, determine a decay lifetime for ortho-positronium (o-Ps) based on the ratio, and determine the health of the tissue based on the decay lifetime for o-Ps.

Disclosed herein also is a Positron Emission Tomography (PET) scanner. The PET scanner includes a plurality of sensors for detecting an event related to decay of a positron emitted from a pharmaceutical radionuclide in the tissue, and a processor. The processor is configured to measure a first count rate indicative of three photon emission related to a first decay mode of the positron, measure a second count rate indicative of two photon emission related to a second decay mode of the positron, apply a scatter correction factor to each of the first count rate and the second count rate to account for scattered and attenuated annihilation photons in the tissue, determine a ratio of the first count rate to the second count rate, determine a decay lifetime for ortho-positronium (o-Ps) based on the ratio, and determine the health of the tissue based on the decay lifetime for o-Ps.

### BRIEF DESCRIPTION OF THE DRAWINGS

These and other features, aspects, and advantages of the present invention will become better understood with reference to the following description and appended claims, and accompanying drawings where:

FIG. 1 shows a Positron Emission Tomography scanner in an illustrative embodiment;

FIG. 2 shows a chart depicting various modes of decay for a positron;

FIG. 3 shows spin states of para-positronium (p-Ps);

FIG. 4 shows spin states of ortho-positronium (o-Ps);

FIG. 5 is a three-dimensional grid depicting a 3-photon decay of o-Ps, in an illustrative embodiment;

FIG. 6 shows a relation between measured o-Ps decay lifetime and o-Ps decay rate for several materials; and

FIG. 7 shows a flowchart of a method for measuring o-Ps decay due to a pharmaceutical radiotracer injected in the tissue.

It should be understood that the various embodiments are not limited to the arrangements and instrumentality shown in the drawings.

### DETAILED DESCRIPTION OF THE INVENTION

The present invention may be understood more readily by reference to the following detailed description of preferred embodiments of the invention as well as to the examples included therein. All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may include numbers that are rounded to the nearest significant figure.

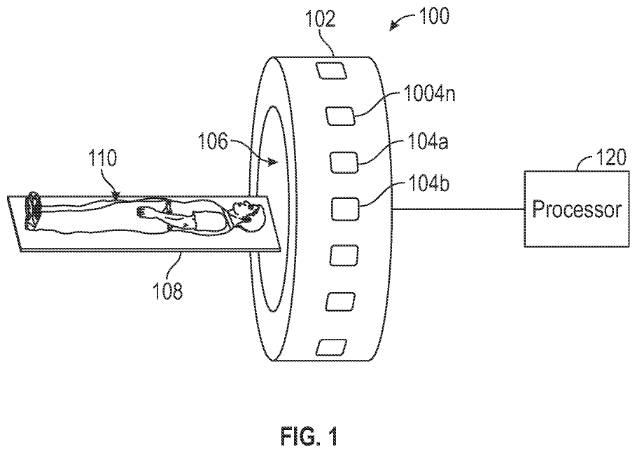

FIG. 1 shows a Positron Emission Tomography scanner (PET scanner 100) in an illustrative embodiment. The PET scanner 100 includes a toroidal housing 102 having a plurality of sensors 104a-n disposed circumferentially around a hole 106 formed by the toroidal housing 102. A platform 108 can be moved into and out of the hole 106 and a person 110, or another organism, lies on the platform 108 to be placed in the PET scanner 100. The person 110 is given an injection of a positron-emitting pharmaceutical radionuclide, such as fluorodeoxyglucose (FDG). The pharmaceutical radionuclide is generally absorbed at a higher rate in any cancerous tissues in the person 110. The pharmaceutical radionuclide emits positrons, and the sensors 104a-n measure the annihilation photons emitted by the various modes of decay of the positrons. A processor 120 receives data from the sensors 104a-n and calculates various quantities discussed herein, which are used to assess tissue health in person 110 or gain additional diagnostic information for a suspected cancer lesion such as if the lesion is hypoxic.

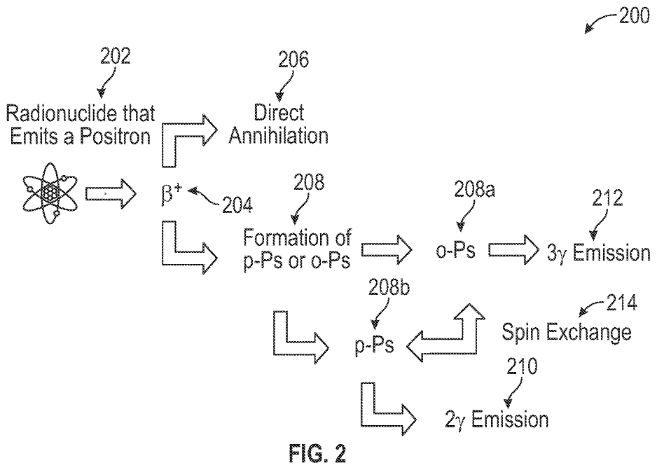

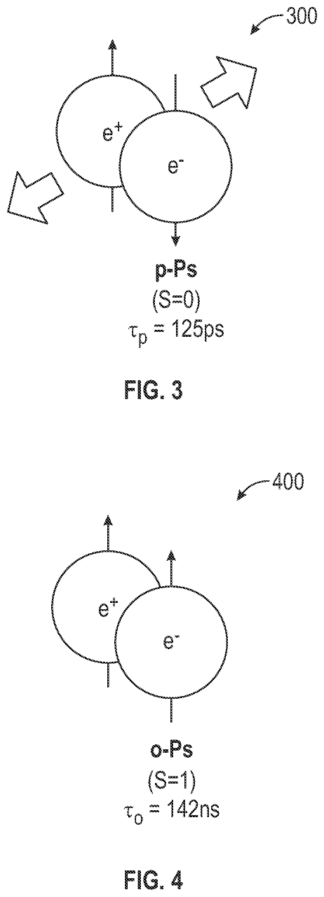

FIG. 2 shows a chart 200 depicting various modes of decay for a positron. The decay process of chart 200 starts at a radionuclide 202 that, for illustrative purposes, has been injected into the person. The radionuclide 202 emits a positron 204. The positron 204 can decay by direct annihilation 206 with a free electron, which results in the emission of two photons (2-photon emission), which are detected at the PET scanner 100. Alternatively, the positron 204 can bond with a free electron to form positronium (Ps) 208. The Ps 208 can take the form of either ortho-positronium (o-Ps) or para-positronium (p-Ps). As shown in FIG. 3, p-Ps includes the positron 206 and the electron 302 with anti-parallel spin states (e.g., +½, −½). The p-Ps has a decay lifetime of 125 picoseconds (τₚ=125 ps) in a vacuum. As shown in FIG. 4, o-Ps includes the positron 206 and the electron 302 with parallel spin states (e.g., +½, +½). The o-Ps has a decay lifetime of 142 nanoseconds (to =142 ns) in a vacuum.

Referring again to FIG. 2, p-Ps 208b can convert to o-Ps 208a, and vice-versa, through a spin-exchange interaction 214. The o-Ps 208b primarily decays via 2 photon emission 210 which is detected by the PET scanner 100. The o-Ps 208a primarily decays via the emission of three photons (three-photon emission 212) which are detected by the PET scanner 100.

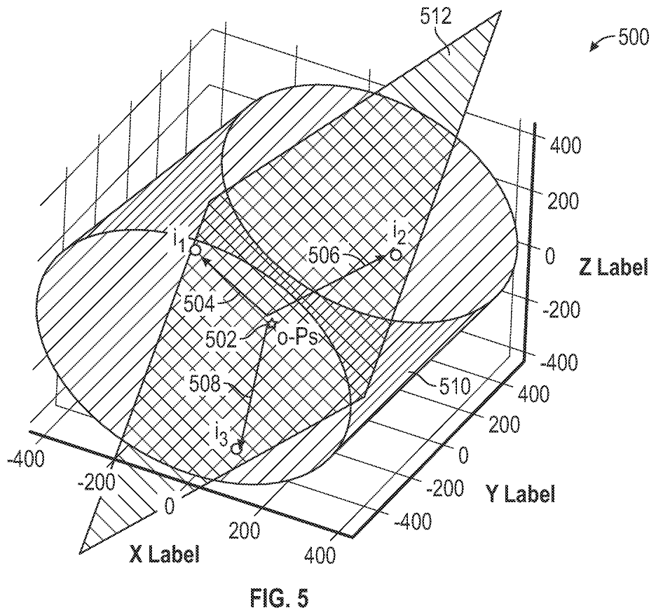

FIG. 5 is a three-dimensional grid 500 depicting a 3-photon decay of o-Ps in an illustrative embodiment. The o-Ps is located at a source location 502. Decay of the o-Ps generates three photons 504, 506 and 508 which are detected as events i₁, i₂ and i₃, respectively, at the PET scanner 100 (represented by blue cylinder 510).

Conversation of energy and conversation of momentum can be applied to the events i₁, i₂, i₃ to classify the three-photon event as an o-Ps decay. Various criteria are imposed on the events i₁, i₂, i₃ to identify the presence of the o-Ps decay. One criterion is that each event (i₁, i₂, i₃) has an energy that is less than 550 keV and the sum of the energies of the events is in a range of 1022+/−100 keV. Another criterion is that the plane of interaction (i.e., plane 512) that contains the three photons 504, 506, 508 contains the source location 502. Alternatively, spherical back-projections of the photons 504, 506, 508 should intersect at a same location (within a selected criterion). This same location is then identified as the source location 502. Another criterion is that emission angles between the photons 504, 506, 508 are less than 170° and greater than 10°. Another criterion is that the photons 504, 506, 508 are emitted within 100 ps of each other.

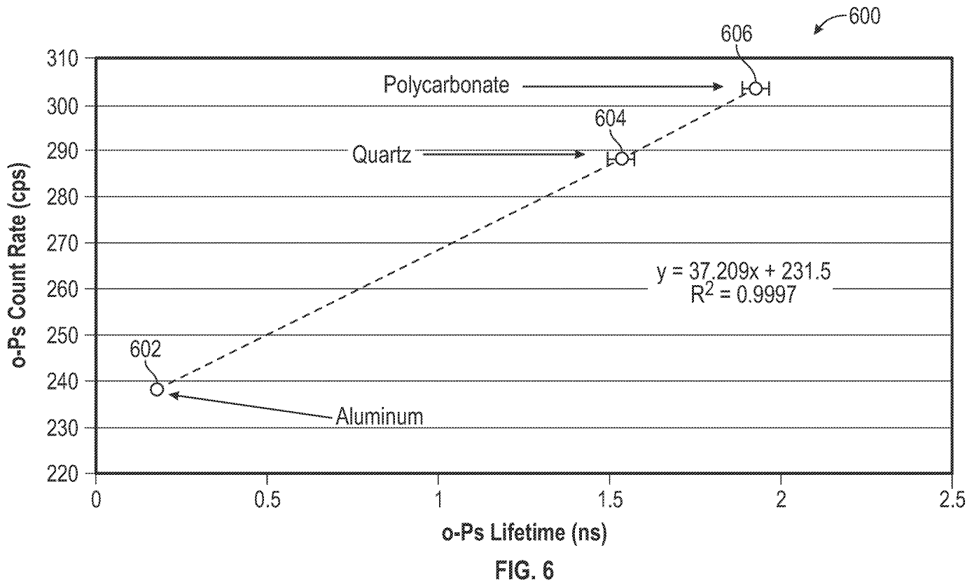

FIG. 6 shows a relation 600 between measured o-Ps decay lifetime and o-Ps decay rate. The o-Ps decay lifetime is shown along the abscissa in nanoseconds (ns). The o-Ps count rate is shown along the ordinate axis in counts per second (cps). Data points are shown for aluminum 602, quartz 604, and polycarbonate 606. The relation 600 shows that o-Ps decay lifetime changes with the material in which the ortho-positron resides when it decays. Therefore, a relation such as shown in FIG. 6 can be used with the decay lifetime to determine a type of tissues and the health of the tissue.

For example, hypoxic liver tissue has a dissolved oxygen concentration of about 6 mmHg while healthy liver tissue has a dissolved oxygen concentration of about 40 mmHg. Oxygen interacting with Ps can cause the Ps to undergo spin exchange 214. The hypoxic liver tissue will exhibit a different o-Ps lifetime relative to healthy liver tissue due to the spin exchange from oxygen. The lifetime of o-Ps decay can therefore be used to determine a concentration of oxygen in the liver tissue and thereby determine whether the liver tissue is hypoxic or healthy.

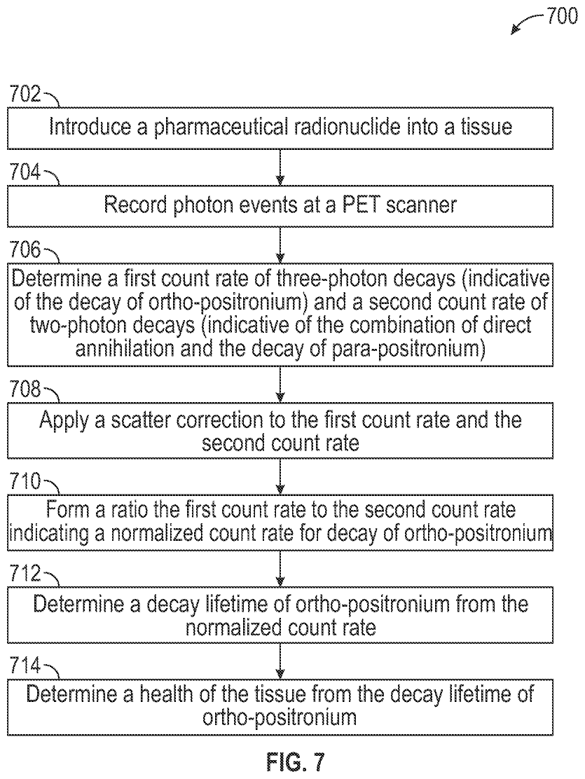

FIG. 7 shows a flowchart 700 of a method for determining a type of tissue and the health of the tissue from a detection of o-Ps decay due to a pharmaceutical radionuclide injected into a patient. The method begins at box 702, in which a pharmaceutical radionuclide (e.g., FDG) is introduced into the person and the person is placed in the PET scanner 100. The pharmaceutical radionuclide tends to be absorbed at a greater rate in cancerous tissue and not absorbed (or is absorbed to a lesser degree) by non-cancerous tissue. The pharmaceutical radionuclide emits positrons, which decay in the various modes discussed with respect to FIG. 2.

In box 704, photon emission events are recorded at the PET scanner. In box 706, the recorded events are processed in the processor to determine a first count rate for three-photon emissions as well as a second count rate indicative of the number of two-photon emissions. The first count rate is indicative of the decay of o-Ps. The second count rate is indicative of the combination of direct annihilation and the decay of p-Ps. In box 708, a scatter correction is applied to the first count rate and the second count rate to account for attenuation and scatter of annihilation photons due to interaction within the body/tissue/patient. In box 710, a ratio is formed of the (corrected) first count rate to the (corrected) second count rate. The ratio provides a normalization of the first count rate from which the rate of decay for ortho-positronium can be determined. In box 712, a decay lifetime for ortho-positronium is determined from the rate of decay. In box 714, a tissue composition is determined from the decay lifetime of o-Ps. Thus, health of a tissue is determined from the decay lifetime of o-Ps.

In various embodiments, the processor 120 can run a machine learning program that determines a health of a tissue, an oxygen concentration and/or an amount of hypoxic tissue based on a decay lifetime for o-Ps, as disclosed herein. The machine learning program can be a neural network. The program can be trained using a known set of data or using one or more tissues having known compositions or known levels of hypoxia, etc. The trained program can receive decay data from the sensors of the scanner related to positron decay at a tissue being tested and output the health of the tissue being tested.

Although the present invention has been described in considerable detail with reference to certain preferred versions thereof, other versions are possible. Therefore, the spirit and scope of the appended claims should not be limited to the description of the preferred versions contained herein.

The reader's attention is directed to all papers and documents which are filed concurrently with this specification, and which are open to public inspection with this specification, and the contents of all such papers and documents are incorporated herein by reference.

All the features disclosed in this specification (including any accompanying claims, abstract, and drawings) may be replaced by alternative features serving the same, equivalent or similar purpose, unless expressly stated otherwise. Thus, unless expressly stated otherwise, each feature disclosed is one example only of a generic series of equivalent or similar features.

Any element in a claim that does not explicitly state “means for” performing a specified function, or “step for” performing a specific function, is not to be interpreted as a “means” or “step” clause as specified in 35 U.S. C § 112, sixth paragraph. In particular, the use of “step of” in the claims herein is not intended to invoke the provisions of 35 U.S. C § 112, sixth paragraph.

What is claimed is:

1. A method of measuring a health of a tissue, comprising:
introducing a pharmaceutical radionuclide into the tissue, wherein the pharmaceutical radionuclide emits a positron;
measuring a first count rate indicative of three photon emission related to a first decay mode of the positron;
measuring a second count rate indicative of two photon emission related to a second decay mode of the positron;
applying a scatter correction factor to each of the first count rate and the second count rate to account for scattered and attenuated annihilation photons in the tissue;
determining a ratio of the first count rate to the second count rate;
determining a decay lifetime for ortho-positronium (o-Ps) based on the ratio; and
determining the health of the tissue based on the decay lifetime for o-Ps.

2. The method of claim 1, wherein the pharmaceutical radionuclide is fluorodeoxyglucose (FDG) containing ¹⁸F.

3. The method of claim 1, further comprising determining an amount of the pharmaceutical radionuclide in the tissue and determining the health of the tissue based on the decay lifetime of the o-Ps in the tissue.

4. The method of claim 1, further comprising determining an oxygen concentration in the tissue from the decay lifetime of the o-Ps.

5. The method of claim 4, further comprising determining an amount of hypoxic tissue from the oxygen concentration.

6. The method of claim 1, further comprising detecting three events and determining the occurrence of the three-photon emission when the three events meet one or more criteria.

7. The method of claim 6, wherein the one or more criteria includes at least one of:
(i) each event is less than 550 keV; (i) the sum of the energies of the events is in a range of 1022+/−100 keV; (iii) a plane of interaction of the photons intersect at a source location; (iv) an emission angle between the photons is less than 170° and greater than 10°; and (v) photons are emitted within 100 picoseconds of each other.

8. The method of claim 1, wherein the first decay mode is the decay of o-Ps.

9. The method of claim 1, wherein the second decay mode includes at least one of a decay of p-Ps and direct annihilation of the positron.

10. A device for measuring the health of a tissue, comprising:
a plurality of sensors for detecting an event related to decay of a positron emitted from a pharmaceutical radionuclide in the tissue;
a processor configured to:
measure a first count rate indicative of three photon emission related to a first decay mode of the positron;
measure a second count rate indicative of two photon emission related to a second decay mode of the positron;
apply a scatter correction factor to each of the first count rate and the second count rate to account for scattered and attenuated annihilation photons in the tissue;
determine a ratio of the first count rate to the second count rate;
determine a decay lifetime for ortho-positronium (o-Ps) based on the ratio; and
determine the health of the tissue based on the decay lifetime for o-Ps.

11. The device of claim 10, wherein the processor is further configured to operate a machine learning program to determine the health to the tissue based on the decay lifetime for o-Ps.

12. The device of claim 10, wherein the processor is further configured to determine an amount of the pharmaceutical radionuclide in the tissue and determine the health of the tissue based on the decay lifetime of the o-Ps in the tissue.

13. The device of claim 10, wherein the processor is further configured to determine an oxygen concentration in the tissue from the decay lifetime of the o-Ps.

14. The device of claim 13, wherein the processor is further configured to determine a region of hypoxic tissue from the oxygen concentration.

15. The device of claim 10, wherein the plurality of sensors is further configured to detect three events and the processor is further configured to determine the occurrence of the three-photon emission when the three events meet one or more criteria.

16. The device of claim 15, wherein the one or more criteria includes at least one of: (i) each event is less than 550 keV; (i) the sum of the energies of the events is in a range of 1022+/−100 keV; (iii) a plane of interaction of the photons intersect at a source location; (iv) an emission angle between the photons is less than 170° and greater than 10°; and (v) photons are emitted within 100 picoseconds of each other.

17. The device of claim 10, wherein the first decay mode is the decay of o-Ps and the second decay mode includes at least one of a decay of p-Ps and direct annihilation of the positron.

18. A Positron Emission Tomography (PET) scanner, comprising:
a plurality of sensors for detecting an event related to decay of a positron emitted from a pharmaceutical radionuclide in the tissue;
a processor configured to:
measure a first count rate indicative of three photon emission related to a first decay mode of the positron;
measure a second count rate indicative of two photon emission related to a second decay mode of the positron;
apply a scatter correction factor to each of the first count rate and the second count rate to account for scattered and attenuated annihilation photons in the tissue;
determine a ratio of the first count rate to the second count rate;
determine a decay lifetime for ortho-positronium (o-Ps) based on the ratio; and
determine the health of the tissue based on the decay lifetime for o-Ps.

19. The PET scanner of claim 18, wherein the processor is further configured to determine an amount of the pharmaceutical radionuclide in the tissue and determine the health of the tissue based on the decay lifetime of the o-Ps in the tissue.

20. The PET scanner of claim 18, wherein the plurality of sensors is further configured to detect three events and the processor is further configured to determine the occurrence of the three-photon emission when the three events meet one or more criteria, wherein the one or more criteria includes at least one of: (i) each event is less than 550 keV; (i) the sum of the energies of the events is in a range of 1022+/−100 keV; (iii) a plane of interaction of the photons intersect at a source location; (iv) an emission angle between the photons is less than 170° and greater than 10°; and (v) photons are emitted within 100 picoseconds of each other.
